(19) United States
(12) Patent Application Publication
(10) Pub. No.: US 2012/0303009 A1
(43) Pub. Date: Nov. 29, 2012

(54) CORNEA CENTER POSITIONING METHOD FOR EXCIMER LASER KERATOMILEUSIS

(75) Inventors: Danghui Liu, Wenzhou (CN); Jia Qu, Wenzhou (CN); Zhaokai Tang, Wenzhou (CN); Hao Chen, Wenzhou (CN); Shihao Chen, Wenzhou (CN)

(73) Assignees: RESEARCH & DEVELOPMENT INSTITUTE OF VISION SCIENCE, WENZHOU MEDICAL COLLEGE, Wenzhou, Zhejiang (CN); ZHEJIANG WENZHOU MEDICAL COLLEGE & LASERSIGHT TECHNOLOGIES CO., LTD., Wenzhou, Zhejiang (CN)

(21) Appl. No.: 13/574,570

(22) PCT Filed: Dec. 7, 2010
(86) PCT No.: PCT/CN2010/079506

§ 371 Date: Jul. 20, 2012

(30) Foreign Application Priority Data
Jan. 22, 2010 (CN) 201010102098.3

(51) Int. Cl. A61F 9/01

(52) U.S. Cl. 606/5

(57) ABSTRACT

A cornea center positioning method for excimer laser keratomileusis includes the following steps of establishing a horizontal offset model and a vertical offset model by way of measuring the pupil diameter and offset of the pupil center relative to the center of the corneal vertex under different luminance levels, and inputting data of the established models into a laser keratomileusis machine with an eye tracking system, which can dynamically track the pupil location and keep the ablation center precisely at the center of the corneal vertex.

### FIELD OF THE INVENTION

The present invention relates to a cornea center positioning method for excimer laser keratomileusis, said method including: establishing a model of a horizontal offset and a model of a vertical offset by way of measuring the pupil diameter and center-shifting offset of the pupil center relative to the center of the corneal vertex under different luminance levels and inputting the data of the established models into a laser keratomileusis machine with an eye tracking system, so as to achieve dynamically tracking of the pupil in the laser keratomileusis.

### BACKGROUND OF THE INVENTION

The pupil, which is a pore surrounded by iris, is an important component in the optical system of the human eye. The main function of the pupil is to maintain the stable arrival of light ray to the fundus under different lighting environments by changing its size. Moreover, the pupil size has a great influence on the focal depth of eye imaging and the aberration of the whole eye.

It is of crucial importance to position and maintain the corneal ablation center during the laser keratomileusis, especially during the laser keratomileusis directed by aberration. In order to facilitate the operation, it is generally assumed that the therapeutic center passes through theoretical visual axis and the corneal vertex during the laser keratomileusis. Generally, an eye tracking system tracks the pupil (pupil center), but the center of pupil differs from the corneal vertex. In certain cases, such difference can be very significant. To solve this problem, the current method is to introduce a fixed center-shifting offset to compensate this difference. That is, the center of the pupil is tracked, but the therapeutic region is centered on the corneal vertex which is close to the visual axis. However, many conditions may change during the surgical operation: the size of pupil may change with the surrounding illumination, the difference of light ray within eyes caused by the direction of watching may also affect the size of the pupil, and the pupil may dilate due to stimulation of sympathetic nerve caused by stress, and the change of modulating state will also change the size of the pupil. Thus the change of the pupil size will change the position of pupil center.

Some studies in the prior art show that the position of pupil center will vary with the pupil size. Fay, et al. (1992) shows the variation of the pupil center during mydriasis can be up to 0.7 mm. The study of Wilson, et al. (1992) on the pupil size and its central position under 5 different illumination conditions has shown that the central position of the pupil changes with the size of the pupil with the maximal variation of 0.6 mm, and there is a linear relationship between the variation of the central position of the pupil and the size of the pupil in approximately half of the subjects. In addition, variations in left and right eyes are symmetric. Studies in China ^{[4]} on the pupil center in the Lasik have shown that the position of pupil center all changed during surgeries in 203 cases for 394 eyes, with 0.18 mm of the horizontal offset and 0.16 mm of the vertical offset in right eyes as well as 0.31 mm of the horizontal offset and 0.11 mm of the vertical offset in left eyes. These suggest that the pupil centers of the two eyes changes significantly during the Lasik. The study of Yang, et al. (2002) on the position of pupil centers in 70 subjects under conditions of scotopic, mesopic and photopic visions and mydriasis shows that the average offset variation is 0.133 mm, with the maximal variation of 0.6 mm. Bara ^{[6]} suggests that if the shift of pupil center is neglected, it may cause undesirable results in the laser keratomileusis directed by wavefront aberration. The study of Porter, et al. (2006) on laser keratomileusis directed by aberration shows that the variation of pupil center caused by drug-induced mydriasis may reach 0.29±0.14 mm, which caused an increase of higher-order aberrations in the operated eye, and thus affecting the quality of visual sense.

These results suggest that the laser system using fixed center-shifting offset can cause ablation error in laser surgeries, and such error may be very significant in some cases. In particular, in the corneal laser surgeries, the illumination is about 600-2000 lux, which differs greatly from 130-300 lux generally for the lightning in indoor inspection, and therefore, the pupil size and the central position of the pupil during operations are different from those in the natural state. Accordingly, we have to study the position of pupil center relative to the center of the corneal vertex for different pupil sizes under different illumination circumstances, to establish mathematical models of the relationship between pupil size and the central position of the pupil, in order to employ them in clinical applications of keratomileusis in future for the offset corrections in corneal ablations.

### SUMMARY OF THE INVENTION

### Technical Problems of the Invention

The purpose of the present invention is to overcome the inadequacies in the prior art as well as to provide a cornea center positioning method for excimer laser keratomileusis, which is capable of determining the center of corneal vertex precisely.

Technical Solutions of the Invention

To achieve the above-mentioned purpose, the present invention discloses a cornea center positioning method for excimer laser keratomileusis, characterized by including the following steps:

1) to set an illuminating brightness;

2) to obtain images of Placido's disc and the pupil simultaneously by using instruments;

3) to record the size of pupil and the position of pupil center relative to the center of the vertex in the corneal topography;

4) to calculate and record the horizontal center-shifting offset X and vertical center-shifting offset Y;

5) to change the illuminating brightness, and to repeat step 2) to 4) for twice or more;

### EFFECT OF THE INVENTION

The advantageous effects of the present invention in comparison with the prior art is as follows: the method according to the present invention comprises establishing a model of a horizontal offset and a model of a vertical offset by way of measuring the pupil diameter and offset of the pupil center relative to the center of the corneal vertex under different luminance levels, and inputting data of the established models into a laser keratomileusis machine with an eye tracking system, resulting in dynamically tracking of the pupil location in the laser keratomileusis, and thus reducing the error of the tracking system, significantly increasing the visual quality following the laser keratomileusis.

The present invention will be further described in combination with the accompanying drawings and examples.

### DESCRIPTION OF THE DRAWINGS

FIG. 1 is the schematic diagram of the relationship between the pupil, the ablation area, the pupil center, the ablation center, and the center-shifting offset;

FIG. 2 is the schematic diagram for simultaneously obtaining the image of the eye, the Placido's disc, and the pupil (front view) by AstraMax;

FIG. 3 is the pupil image collected by AstraMax;

FIG. 4 is the overlay diagram of the pupil diameter and the center-shifting offset of the pupil center relative to the center of the corneal vertex ;

FIG. 5 is the schematic diagram of the high order polynomial model-horizontal center-shifting offset model (the curve of the horizontal center-shifting offset versus the pupil diameter);

FIG. 6 is the schematic diagram of linear model-vertical center-shifting offset model (the curve of the vertical center-shifting offset versus the pupil diameter);

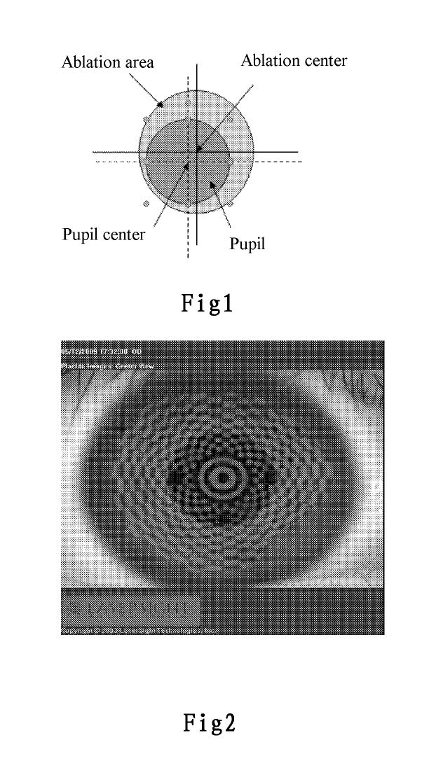

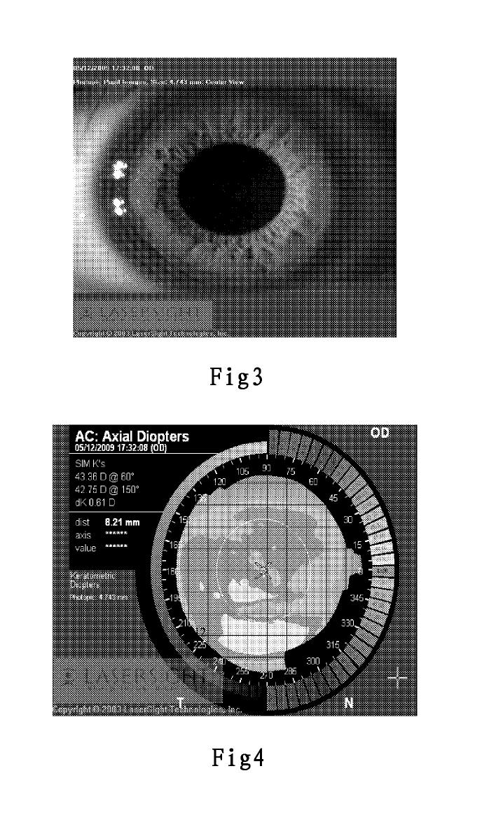

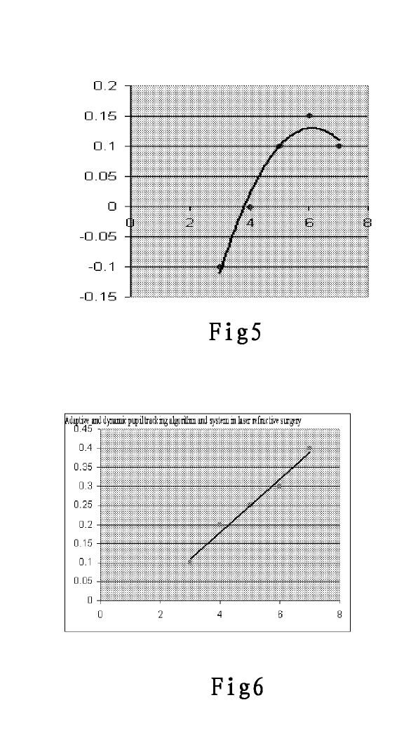

### THE EMBODIMENTS OF THE INVENTION

In this example, the apparatus is selected as AstraMax 3D Corneal Topography System manufactured by Lasersight Technolgies, Inc. for obtaining images. One advantage of this apparatus is the ability to customize the capture, and it can simultaneously obtain images of the eye, the Placido's disc and the pupil, as well as can change the illumination level so as to stimulate the tested eye to change the pupil size. The set value of illumination was 0-255, which is suitable for the application in the method according to the present invention. The illuminating system of the AstraMax 3D Corneal Topography System comprises Placido illuminating optotype with a wavelength of 660 nm, and illumination for infrared with a wavelength of 875 nm. Two or more illumination levels are set as sampling points and the more sampling points are set, the more precise it will be. Generally, 4-10 illumination levels are set as sampling points, and 6 illumination levels were set as sampling points in this example. According to the Weber-Fechner law, if an external stimulus varies as a geometric progression, the corresponding perception is altered in an arithmetic progression. Therefore, in the division of illumination levels of the 6 sampling points in this example, the principle of exponent movement is employed, in which 0-255 is divided into 6 intervals, i.e. 255, 92, 67, 56, 48, 44, 0, which respectively correspond to the plane illumination of 355, 133, 50, 18.8, 7.1, 2.66, 0 lux. AstraMax 3D Corneal Topography System was used to test under the illumination levels of 355, 133, 50, 18.8, 7.1, 2.66, 0 lux respectively for several times, to obtain the corresponding images of the eye, the Placido's disc and the pupil. The horizontal center-shifting offset X and vertical center-shifting offset Y of the pupil center relative to the center of the vertex in the corneal topography were calculated and recorded. Then, the curves of the horizontal center-shifting offset X and the vertical center-shifting offset Y versus the pupil diameter were drawn based on the data as obtained, respectively. The models were established using 2-order or 3-order polynomials, i.e.

Horizontal center-shifting model:

offsetX=a₀+a₁d+a₂d²+ . . . (1)

Vertical center-shifting model:

offsetY=b₀+b₁d+b₂d²+ . . . (2)

The horizontal center-shifting model and the vertical center-shifting model were then input into a laser machine, and then the pupil size and the central position of the eye in a patient were determined using the eyeball tracking system of the laser machine. Finally, the data for precise positions of the corneal vertex were obtained according to the obtained pupil size and the data for central positions in combination with the horizontal center-shifting model and the vertical center-shifting model.

During the operation and the eye tracking, the data for the pupil diameter and the position were obtained. Dynamically variable horizontal center-shifting offset and vertical center-shifting offset were calculated by the horizontal center-shifting and vertical center-shifting models using immediate data of the pupil diameter, respectively. The above dynamically variable horizontal and vertical center-shifting offsets would be used for compensating the change of pupil center relative to the center of the corneal vertex, and that is, the position of the pupil is dynamically variably tracked and the ablation center at the center of the corneal vertex (visual axis) is precisely maintained.

In the present invention, dynamically tracking of the cornea in the laser keratomileusis is achieved by way of measuring the pupil diameter and offset of the pupil center relative to the center of the corneal vertex under different luminance levels, establishing a model of a horizontal offset and a model of a vertical offset, and inputting data of the established models into a laser keratomileusis machine with an eye tracking system, resulting in dynamically tracking of the pupil location in the laser keratomileusis, and thus reducing the error of the tracking system, significantly increasing the visual quality following the laser keratomileusis.

1. A cornea center positioning method for excimer laser keratomileusis, characterized by including the following steps:
1) to set an illuminating brightness;
2) to obtain Placido's disc and images of the pupil simultaneously by using an instrument;
3) to record the size of pupil and the position of pupil center relative to the center of the vertex in the corneal topography;
4) to calculate and record the horizontal center-shifting offset X and vertical center-shifting offset Y relative to the center of the vertex in the corneal topography;
5) to change the illuminating brightness, and to repeat step 2) to 4) for twice or more times;
6) to draw the curves of the horizontal center-shifting offset X and the vertical center-shifting offset Y versus the pupil diameter according to the data as obtained, respectively;
7) to establish an model of horizontal center-shifting and an model of vertical center-shifting using 2-order or 3-order polynomials, respectively;
8) to input the model of horizontal center-shifting and the model of vertical center-shifting into a laser machine;
9) to determine the pupil size and the central position of the eye in a patient using the eyeball tracking system of the laser machine;
10) to obtain the data for the precise position of the corneal vertex according to the obtained data for the pupil size and the central position in combination with the horizontal center-shifting model and the vertical center-shifting model.

2. The cornea center positioning method for excimer laser keratomileusis according to claim 1, characterized in that: the illuminating brightness is 0, 44, 48, 56, 255.
